(19) United States
(12) Patent Application Publication
(10) Pub. No.: US 2014/0276583 A1
(43) Pub. Date: Sep. 18, 2014

(54) INJECTION DEVICE WITH AUTOMATIC DATA CAPTURE AND TRANSMISSION

(71) Applicant: Bayer Healthcare LLC, Whippany, NJ (US)

(72) Inventors: Jun Chen, Warren, NJ (US); Igor Gofman, Croton-on-Hudson, NY (US); Mu Wu, Hopewell Junction, NY (US); James Johnson, Danbury, CT (US); Qiang Fu, Briarcliff Manor, NY (US); Simin Yao, Boonton Twp., NJ (US)

(21) Appl. No.: 14/206,382

(22) Filed: Mar. 12, 2014

Related U.S. Application Data
(60) Provisional application No. 61/791,148, filed on Mar. 15, 2013.

(51) Int. Cl. A61M 5/315

(52) U.S. Cl. CPC A61M 5/31546; A61M 5/31573
USPC 604/506; 604/207

(57) ABSTRACT

An injection device, which can be, e.g., an insulin pen, can capture and transmit injection and/or medicament information to another device, such as, e.g., a blood glucose meter. The injection and medicament information can include, e.g., time and date of injection, type of medicament injected, and dosage amount. The injection device can include a dial cap that can be rotated to select a dosage amount to be injected. In some embodiments, the dial cap can include a microcontroller with transmitter, a Hall effect sensor, and a ring magnet. In some embodiments, the injection device can include a medicament cartridge with a 1-wire EEPROM, which can have medicament information stored therein. Methods of operating an injection device are also provided, as are other aspects.

### CROSS REFERENCE TO RELATED APPLICATION

This claims the benefit of U.S. Provisional Patent Application No. 61/791,148, filed Mar. 15, 2013, and entitled “Injection Device With Automatic Data Capture And Transmission” (Attorney Docket BHC124022US (BHDD/039/L)), which is hereby incorporated herein by reference in its entirety for all purposes.

### FIELD

The invention relates generally to injection devices that deliver a dose of medicament to an injection site, and more particularly to insulin pens that deliver a dose of insulin to an injection site.

### BACKGROUND

An insulin pen is a widely used injection device that allows users to self-administer a pre-determined dose of insulin for the treatment of diabetes. Injection devices such as insulin pens are usually easy to use, accurate, and discreet compared to syringes and vials. Insulin pens typically have a dial that a user can adjust to select the dose of insulin to be delivered by the insulin pen. In some cases, however, determining the correct dose and type of insulin to inject may be difficult without first performing a complex calculation. For example, a bolus calculator, which may be included in a blood glucose meter or other device, can calculate a recommended dose of insulin based on several parameters that can include the time, amount, and type of a previous injected dose. However, accurately recording the time, amount, and type of each injected dose may not always be possible for some users. Therefore, a need exists to provide an injection device that can automatically transmit relevant information regarding an injection to another device that can record the information and/or perform recommended dose calculations.

### SUMMARY

According to one aspect, a dial cap configured to be attached to a housing of an injection device is provided. The dial cap comprises a rotatable portion configured to rotate to select a dosage amount to be delivered by the injection device, a Hall effect sensor configured to rotate with the rotatable portion and to output a voltage proportional to an angle of rotation of the Hall effect sensor, a magnetic ring encircling the Hall effect sensor, and a microcontroller. The microcontroller is configured to receive the output voltage of the Hall effect sensor, determine a dosage amount in response to receiving the output voltage of the Hall effect sensor, and transmit a determined dosage amount.

According to another aspect, a medicament cartridge configured to be received in a cartridge container of an injection device is provided. The medicament cartridge comprises a hollow cylindrical body configured to contain a medicament therein and having an open end, a piston configured to seal the open end, and an end cap comprising a 1-wire EEPROM (electrically erasable programmable read only memory) configured to store medicament information therein, the end cap configured to fit over the open end.

According to a further aspect, a method of operating an injection device is provided. The method comprises receiving an output voltage from a Hall effect sensor located in the injection device, determining an amount of a dosage based on the received output voltage, receiving medicament information from a 1-wire EEPROM located in a medicament cartridge of the injection device, and transmitting the determined amount of the dosage and the medicament information to a device external to the injection device.

Still other aspects, features, and advantages of the invention may be readily apparent from the following detailed description wherein a number of example embodiments and implementations are described and illustrated, including the best mode contemplated for carrying out the invention. The invention may also include other and different embodiments, and its several details may be modified in various respects, all without departing from the scope of the invention. Accordingly, the drawings and descriptions are to be regarded as illustrative in nature, and not as restrictive. The drawings are not necessarily drawn to scale. The invention covers all modifications, equivalents, and alternatives falling within the scope of the invention.

### BRIEF DESCRIPTION OF DRAWINGS

The skilled artisan will understand that the drawings, described below, are for illustration purposes only. The drawings are not intended to limit the scope of this disclosure in any way.

FIG. 1 illustrates a side view of an injection device with a portion of the housing removed to show internal components according to the prior art.

FIGS. 2A and 2B illustrate a cross-sectional front view and a left side view, respectively, of a medicament cartridge according to the prior art.

FIG. 3 illustrates a front view of a dial cap according to embodiments;

FIG. 4 illustrates a partial cross-sectional front view of an injection device incorporating the dial cap of FIG. 3 according to embodiments.

FIG. 5 illustrates a plan view of a ring magnet used in the injection device of FIG. 4 according to embodiments.

FIGS. 6A and 6B illustrate a top view and an exploded cross-sectional front view (taken along line 6B-6B of FIG. 6A), respectively, of a medicament cartridge according to embodiments.

FIG. 7 illustrates a simplified front view of an injection device having the medicament cartridge of FIG. 6 received therein according to embodiments.

FIG. 8 illustrates a block diagram of an injection device according to embodiments.

FIG. 9 illustrates a flowchart of a method of operating an injection device according to embodiments.

### DETAILED DESCRIPTION

Reference will now be made in detail to the example embodiments of this disclosure, which are illustrated in the accompanying drawings. Wherever possible, the same reference numbers will be used throughout the drawings to refer to the same or like parts.

In one aspect, an injection device can include a dial cap having a Hall effect sensor and a microcontroller for determining a dosage amount injected by the injection device and for transmitting that information along with other relevant information, such as, e.g., time and date of the injection, to another device for recording and/or processing. For example, in the case of insulin injections, the other device can be a blood glucose meter that includes a bolus calculator. In some embodiments, the injection device can include a medicament cartridge that has a 1-wire EEPROM (electrically erasable programmable read only memory) incorporated therein. Medicament information, such as, e.g., the type of medicament, the expiration date of the medicament, the manufacturer, etc., can be stored in the 1-wire EEPROM. The stored medicament information can be read by the microcontroller and transmitted along with the injection information after each injection. This can relieve a user, such as, e.g., a diabetic user who regularly injects insulin, from the tedious and time-consuming task of manually recording injection and medicament information after each injection. In other aspects, methods of operating an injection device are provided, as will be explained in greater detail below in connection with FIGS. 1-9.

FIG. 1 illustrates an example of a known injection device 100, which can be, e.g., an insulin pen. Injection device 100 can include a dial cap 102, a housing 104, and a cartridge container 106. Dial cap 102 can have a rotatable portion 108 configured to rotate (horizontally as shown by arrows) about axis 110 to allow a user to select a dosage to be delivered to an injection site by injection device 100. Housing 104 can be a hollow, cylindrically-shaped structure having a first end 103 and a second end 105. Dial cap 102 can attach to and/or mount over first end 103 and cartridge container 106 can attach to and/or mount over second end 105, each in any suitable manner (e.g., via a friction fit and/or with adhesive). Housing 104 can include therein an engaging mechanism 112 and a plunger 114 having a threaded shaft 116. Threaded shaft 116 can be threadingly-engaged with engaging mechanism 112 and can be mechanically coupled to rotatable portion 108 via, e.g., a connecting shaft (not shown) attached internally to rotatable portion 108. Housing 104 can also include therein a stored energy source which, in some devices, can be a helical spring 118 (shown in an uncompressed state). Housing 104 can further include a release trigger 120 configured to initiate an injection by releasing the energy in the stored energy source (e.g., releasing compressed helical spring 118) to drive plunger 114 through second end 105 and into cartridge container 106.

FIGS. 2A and 2B illustrate an example of a known medicament cartridge 200 that can be used with injection device 100. Medicament cartridge 200 can include a medicament chamber 222, which can contain a medicament, such as, e.g., insulin. Medicament cartridge 200 can contain multiple doses of a medicament and can be replaced in an injection device when the medicament in medicament chamber 222 has been exhausted. Medicament cartridge 200 can have a needle end 223 configured to receive a needle assembly (not shown). A needle assembly can include a needle and needle hub configured to fit over needle end 223. After each injection wherein one or more doses of medicament remain in medicament chamber 222, the used needle assembly can be discarded and replaced with a new, unused needle assembly. Medicament cartridge 200 can also have an open end 225 sealed with a slidable piston 224. Medicament cartridge 200 can be received in medicament cartridge container 106 by inserting open end 225 into open end 107 of medicament cartridge container 106.

Returning to FIG. 1, injection device 100 can be operated as known in the art as follows: a user can insert medicament cartridge 200 into cartridge container 106, and then select a dosage amount to inject by rotating rotatable portion 108 until dosage indicator 126 is aligned with a desired amount (e.g., a number of units), as indicated in dosage display 128. Upon rotation of rotatable portion 108, helical spring 118 can also be rotated and consequently compressed against stop 130. Rotation of rotatable portion 108 to a desired dosage also sets the distance to which plunger 114 and threaded shaft 116 will extend outward from an initial base position (e.g., a zero distance) relative to engaging mechanism 112. Upon placement of injection device 100 against a suitable injection site (e.g., a user's arm or leg), release trigger 120 can be actuated (e.g., by depressing or sliding release trigger 120), which can release compressed helical spring 118. The release of compressed helical spring 118 can drive engaging mechanism 112 and, in turn, plunger 114 the preset distance corresponding to the selected dosage through second end 105 and into medicament cartridge 200, where plunger 114 contacts and drives piston 224 toward needle end 223 in medicament chamber 222. This can cause the selected dosage amount to be injected through the needle (not shown) into the injection site. After injection, rotatable portion 108 can return to an initial position (e.g., zero units). Also, to take into account the amount of medicament injected, the previously set plunger distance becomes the new base position from which subsequent plunger distances corresponding to selected dosage amounts are set.

FIG. 3 illustrates a dial cap 302 in accordance with one or more embodiments. Dial cap 302 can be configured to attach to a housing of an injection device in any suitable manner (e.g., via adhesives, mechanical fasteners, friction fit, etc.). Dial cap 302 can include a rotatable portion 308 configured to rotate (horizontally as shown by arrows) about axis 310 to allow a user to select a dosage to be delivered to an injection site by an injection device. Dial cap 302 can include a dosage indicator 326 marked on an outside surface of rotatable portion 308. A dosage display 328 indicating, e.g., a number of units to be injected, can be displayed on a non-rotatable portion 309 of dial cap 302. A user can select a dosage amount to be injected by rotating rotatable portion 308 until dosage indicator 326 is vertically aligned (as shown) with a desired dosage amount in dosage display 328 (such as, e.g., 30 units as shown). Dial cap 302 can include internal electronic circuitry that can provide automatic capturing of dosage amounts injected and automatic transmission of that information along with time and date information and, in some embodiments, medicament information, to another device, such as, e.g., a suitably equipped blood glucose meter, for recording and/or processing, as described in connection with FIGS. 4 and 5.

FIG. 4 illustrates a portion of an injection device 400, which can be an insulin pen, in accordance with one or more embodiments. Injection device 400 can include dial cap 302 and a housing 404. Dial cap 302 can be attached to and/or mounted over a first end 403 of housing 404 in any suitable manner (e.g., via adhesives, mechanical fasteners, friction fit, etc.). Housing 404 can be similar or identical to housing 104 of injection device 100 and can include a helical spring 418 and extension structures 412a and 412b of an engaging mechanism, which can be similar or identical to engaging mechanism 112 of injection device 100. In alternative embodiments, helical spring 418 can be replaced by other suitable sources of stored energy, such as, e.g., a cylinder of compressed gas, as is known in the art. Housing 404 can also include a plunger and a plunger shaft (not shown) similar or identical to plunger 114 and threaded shaft 116. Housing 404 can further include a release trigger (not shown) similar or identical to release trigger 120.

Injection device 400 can also include a cartridge container and a medicament cartridge received in the cartridge container (neither shown) similar or identical to cartridge container 106 and medicament cartridge 200.

The mechanical operation of injection device 400 with respect to performing an injection can be similar or identical to that described above for injection device 100. In particular, selecting a dosage amount with dial cap 302, setting spring 418 in a compressed state, setting a distance to which the plunger of housing 404 will extend through housing 404 and into the cartridge container and medicament cartridge upon an injection, actuating the release trigger to initiate an injection, returning the rotatable portion 308 to an initial (e.g., zero dosage) position, and establishing the previously set plunger distance as the new base position for subsequent injections, can each be similar or identical to that described above for injection device 100.

In addition to performing the above-described mechanical dosage selecting function, dial cap 302 can automatically determine a selected dosage amount based on the rotation of rotatable portion 308, and can automatically transmit injection information including, e.g., the determined dosage amount and the time and date of an injection, in response to a user performing an injection. The injection information can be transmitted to a device external to injection device 400, such as, e.g., an appropriately configured blood glucose meter in cases where injection device 400 is an insulin pen.

To automatically determine a dosage amount and automatically transmit injection information, dial cap 302 can include a printed circuit board 432 that, in some embodiments, can be mounted and/or fixedly attached to rotatable portion 308 such that printed circuit board 432 can rotate with rotatable portion 308. Printed circuit board 432 can include a microcontroller 434 and a memory 436 that can be mounted on a first side 433 of printed circuit board 432. Memory 436 can be any suitable non-volatile memory, and microcontroller 434 can include an analog-to-digital converter and a transmitter, along with processing and other suitable circuitry. The transmitter can be, e.g., an RF (radio frequency) transmitter, and/or can be based on ZigBee, BLE (Bluetooth low energy) or ANT wireless technologies. Alternatively, any suitable transmission technology can be used. In some embodiments, microcontroller 434 can be, e.g., Part No. CC2541 F128RHAT by Texas Instruments, Inc., of Dallas, Tex., or Part No. nRF51822 by Nordic Semiconductor ASA of Oslo, Norway, or an equivalent part.

In some embodiments, printed circuit board 432 can also include a Hall effect sensor 438 that can be mounted on a second, opposite side 435 of printed circuit board 432. That is, Hall effect sensor 438 can be mounted on the side of printed circuit board 432 that faces housing 404 (i.e., faces down as shown). Hall effect sensor 438 can be a transducer that varies its output voltage in response to a magnetic field (described in more detail below). In some embodiments, Hall effect sensor 438 can be, e.g., Part No. TCS10DLU by Toshiba America Electronic Components, Inc., of Irvine, Calif., or an equivalent part.

In some embodiments, printed circuit board 432 can be attached to extension structures 412a and 412b. Structures 412a and 412b can rotate with printed circuit board 432 in response to rotation of rotatable portion 308 to set helical spring 418 and the plunger (not shown) of housing 404 for an injection. This can occur similarly or identically as described above in connection with engaging mechanism 112 of injection device 100. Printed circuit board 432 can be attached to extension structures 412a and 412b with screws 440a and 440b, respectively, or, alternatively, in any other suitable manner. In other embodiments, printed circuit board 432 can be attached to one or more other suitable structures of an engaging mechanism in housing 404, provided that rotation of those one or more structures in response to a user selecting a dosage amount with rotatable portion 308 performs a similar or identical function as that of engaging mechanism 112 in setting helical spring 418 and the plunger for an injection.

Dial cap 302 can also include a battery 442 to provide power to printed circuit board 432 and the electrical components mounted thereon including, e.g., microcontroller 434, memory 436, and Hall effect sensor 438. Battery 442 can be, e.g., a CR2032 3-volt lithium cell battery. In alternative embodiments, one or more other batteries of suitable size and type can be used.

Dial cap 302 can further include a ring magnet 444 that can be mounted to housing 404 under printed circuit board 432 as shown in FIG. 4. Ring magnet 444 can remain in a fixed position relative to printed circuit board 432 and rotatable portion 308. That is, ring magnet 444 does not rotate with printed circuit board 432 and rotatable portion 308. Alternatively, ring magnet 444 can be mounted to non-rotatable portion 309 of dial cap 302. As shown in FIG. 5, ring magnet 444 can be donut shaped such that Hall effect sensor 438 (shown in phantom) can be centered within donut hole 546 and thus encircled by ring magnet 444 upon assembly of ring magnet 444 and printed circuit board 432 in dial cap 302. Ring magnet 444 can produce a magnetic field around Hall effect sensor 438. In alternative embodiments, other suitable means of producing a magnetic field around the Hall effect sensor 438 can be used instead of ring magnet 444.

As a user selects a dosage amount by rotating rotatable portion 308 of dial cap 302, printed circuit board 432 and Hall effect sensor 438 also rotate. As Hall effect sensor 438 rotates in the magnetic field produced by ring magnet 444, the output voltage of Hall effect sensor 438 varies in proportion to the angle of rotation. For example, if Hall effect sensor 438 rotates to a 60 degree position, corresponding to a rotation of rotatable portion 308 to a desired dosage amount, a first voltage value can be output by Hall effect sensor 438. If Hall effect sensor 438 rotates to a 90 degree position, a second voltage value can be output. In some embodiments, the initial position of rotatable portion 308 (e.g., zero dosage) can correspond to an output voltage Vmin, while a rotation of 359 degrees can correspond to an output voltage Vmax. Microcontroller 434 can receive the output voltage of Hall effect sensor 438. An analog-to-digital converter in microcontroller 434 converts the received output voltage to a dosage amount. This conversion can be based on programming code stored in memory 436 and executed in microcontroller 434 that can correlate output voltages with dosage amounts. The determined dosage amount can be stored in memory 436. In some embodiments, the return of rotatable portion 308 to its initial (e.g., zero dosage) position after an injection can also be sensed by Hall effect sensor 438, which can output a corresponding voltage value, such as, e.g., Vmin. This can signal microcontroller 434 to automatically transmit injection information (including the determined dosage amount) via an antenna that can be imprinted or otherwise mounted on printed circuit board 432. Note that in some embodiments, rotatable portion 308 can only be rotated by a user in one direction, while rotatable portion 308 can return to its initial position by rotating in the opposite direction. The transmission of injection information can be based on, e.g., ZigBee, BLE (Bluetooth low energy) or ANT wireless technologies.

FIGS. 6A and 6B illustrate a medicament cartridge 600 in accordance with one or more embodiments. Medicament cartridge 600 can include a medicament chamber 622, which can contain a medicament, such as, e.g., insulin. Medicament cartridge 600 can contain multiple doses of a medicament and can be replaced in an injection device when the medicament in medicament chamber 622 has been exhausted. Medicament cartridge 600 can have a needle end 623 configured to receive a needle assembly (see FIG. 7). A needle assembly can include a needle and needle hub configured to fit over needle end 623. After each injection wherein one or more doses of medicament remain in medicament chamber 622, the used needle assembly can be replaced with a new, unused needle assembly. Medicament cartridge 600 can also have an open end 625 sealed with a slidable piston 624. Medicament cartridge 600 can further include an open-ended end cap 648 that can have attached and/or mounted thereto a 1-wire EEPROM (electrically erasable programmable read only memory) 650 that can have medicament information stored therein. In some embodiments, 1-wire EEPROM 650 can be a Part No. DS2431Q by Maxim Integrated, Inc., of San Jose, Calif. The medicament information can include, e.g., the type of medicament, the expiration date of the medicament, the manufacturer, the batch number of the medicament, and/or other information. The 1-wire EEPROM 650 can be attached and/or mounted against an inner surface of side wall 649 of end cap 648 in any suitable manner. In alternative embodiments, other suitable memory devices can be used. End cap 648 can also have a pair of electrical contacts 652a and 652b electrically coupled to 1-wire EEPROM 650 and electrically accessible outside of medicament cartridge 600. The 1-wire EEPROM 650 can require one contact to provide power and serial communications and the other contact to provide a voltage reference/current return (e.g., ground). Electrical contacts 652a and 652b can each have a semi-circular shape and can be attached to and/or mounted on a top ledge-like surface 651 of end cap 648. Electrical contacts 652a and 652b can alternatively have other suitable shapes and can alternatively be attached and/or mounted to end cap 648 in other suitable ways, provided they are electrically accessible outside of medicament cartridge 600. Piston 624 can have a reduced diameter section 627 corresponding to opening 653 in end cap 648. End cap 648 can be configured to fit over open end 625 in any suitable manner. For example, in some embodiments, end cap 648 can be friction fit over and/or glued to open end 625. Alternatively, end cap can be attached to open end 625 in any suitable manner.

Medicament cartridge 600 can be used as a conventional medicament cartridge, such as, e.g., medicament cartridge 200, in injection devices where the plunger of the injection device is appropriately sized to fit through opening 653 of end cap 648. For example, medicament cartridge 600 can be used in injection devices 100 and/or 400 with appropriately sized plungers. Medicament cartridge 600 can also be used in injection devices configured to utilize the information stored in 1-wire EEPROM 650, as described below in connection with FIG. 7.

FIG. 7 illustrates an injection device 700 in accordance with one or more embodiments. Injection device 700, which can be an insulin pen, can include a dial cap 702, a housing 704, a release trigger 720, a cartridge container 706, and medicament cartridge 600 received within cartridge container 706. Dial cap 702 can include electronic circuitry similar or identical to that of dial cap 302 that can automatically capture medicament information and injected dosage amounts and can automatically transmit that information to another device, such as, e.g., a suitably equipped blood glucose meter, for recording and/or processing.

Dial cap 702, which can be similar or substantially identical to dial cap 302, can include a printed circuit board 732 and a microcontroller 734, which can be similar or identical to printed circuit board 432 and microcontroller 434. Microcontroller 734 can include a transmitter, which can be, e.g., an RF (radio frequency) transmitter. Dial cap 702 can also include electrical conductors 754a and 754b electrically connected to printed circuit board 732. A designated one of electrical conductors 754a or 754b can be electrically coupled to an input of microcontroller 734 via connections on printed circuit board 732. The other of electrical conductors 754a or 754b can be coupled to the current return (e.g., ground) of printed circuit board 432. Electrical conductors 754a and 754b, which can be wires, can extend along an inside surface of dial cap 702 and can terminate at respective electrical contacts 756a and 756b at a base 757 of dial cap 702. Electrical conductors 754a and 754b can alternatively extend from printed circuit board 732 to respective electrical contacts 756a and 756b in any suitable manner and can be in any suitable form.

Housing 404 can include electrical conductors 758a and 758b, which can be, e.g., wires. Electrical conductors 758a and 758b can extend from a first end 703 along an inside surface of housing 404 to a second end 705. Electrical conductors 758a and 758b can alternatively extend from first end 703 to second end 705 in any suitable manner and in any suitable form. At first end 703, electrical conductors 758a and 758b can be configured to electrically connect to respective electrical contacts 756a and 756b of dial cap 702. Housing 704 can otherwise be similar or substantially identical to housing 404.

Cartridge container 706 can be configured to receive medicament cartridge 600. Medicament cartridge 600 can have a needle assembly 760 attached thereto, which can include a needle hub 762 and a needle 764. Cartridge container 706 can include electrical contacts 766a and 766b attached and/or mounted in any suitable manner inside an upper end 767 of cartridge container 706. Electrical contacts 766a and 766b can be configured to electrically connect to electrical contacts 652a and 652b of medicament cartridge 600, respectively, upon insertion of medicament cartridge 600 in cartridge container 706. Electrical contacts 766a and 766b can also be configured to electrically connect to respective electrical conductors 758a and 758b of housing 704. Electrical contacts 766a and 766b can be configured in any suitable manner that electrically connects electrical contacts 652a and 652b of medicament cartridge 600 to respective electrical conductors 758a and 758b of housing 704. Cartridge container 706 can be configured to receive medicament cartridge 600 such that the appropriate electrical contact 766a or 766b provides power and data signals to 1-wire EEPROM 650 and the other electrical contact 766a or 766b provides a voltage reference/current return to 1-wire EEPROM 650. Cartridge container 706 can otherwise be similar or substantially identical to cartridge container 106.

Mechanically, injection device 700 can operate similarly or identically as injection devices 100 and/or 400. However, in response to a user initiating an injection by actuating release trigger 720, microcontroller 734 can automatically read the medicament information from 1-wire EEPROM 650 via the aforementioned electrical connections between microcontroller 734 and 1-wire EEPROM 650, and transmit the received medicament information along with injection information (as described above in connection with FIG. 4 and below in connection with FIG. 8) to another device, such as an appropriately configured blood glucose meter in the case of an insulin injection, for recording and/or processing.

FIG. 8 illustrates a block diagram of an injection device 800 in accordance with one or more embodiments. Injection device 800, which can be an insulin pen, can include mechanical components 804, medicament cartridge 600, and a dial 808. Dial 808 can be similar or identical to, e.g., rotatable portion 708 of dial cap 702 or rotatable portion 308 of dial cap 302, and can be used by a user to select a dosage amount to be injected by injection device 800. Dial 808 can be mechanically coupled to mechanical components 804, which can include, e.g., an engaging mechanism, a plunger, a plunger shaft, a helical spring or other stored energy source, a release trigger, and/or any other suitable structures or parts needed to allow a user to select and inject a desired dosage of a medicament from medicament cartridge 600. Dial 808 can also be mechanically coupled to a position sensor 838.

Position sensor 838 can be, in some embodiments, a Hall effect sensor positioned in a magnet field, such as, e.g., Hall effect sensor 438 encircled by ring magnet 444 (see FIGS. 4 and 5). As a user rotates dial 808 to select a dosage, position sensor 838 can correspondingly rotate within the magnetic field. Position sensor 838 can output a voltage proportional to its angle of rotation. For example, a rotation of 30 degrees by position sensor 838 can result in a first output voltage value, while a rotation of 45 degrees by position sensor 838 can result in a second output voltage value. In some embodiments, position sensor 838 can be, e.g., a Hall effect sensor Part No. TCS10DLU by Toshiba America Electronic Components, Inc., of Irvine, Calif., or an equivalent part. In alternative embodiments, position sensor 838 can be an incremental encoder or an absolute position encoder.

Injection device 800 can also include a microcontroller 834 and a memory 836, which can be similar or identical to microcontroller 434 and memory 436 of FIG. 4. Memory 836 can be non-volatile memory and can be used to store programming code executed in microcontroller 834 and data received from medicament cartridge 600 and/or microcontroller 834. Microcontroller 834 can receive output voltage values from position sensor 838, and can include an analog-to-digital converter that can be used to convert the received voltage values to dosage amounts. The conversion from a voltage value to a dosage amount can be based on programming code executed in microcontroller 834 that correlates voltage values with dosage amounts. Microcontroller 834 can also include a transmitter, which can be an RF (radio frequency) transmitter, and/or can be based on ZigBee, BLE (Bluetooth low energy) or ANT wireless technologies. Alternatively, any suitable transmission technology can be used. Microcontroller 834 can be coupled to antenna 868, which can be located on the same printed circuit board on which microcontroller 834 is mounted. In some embodiments, microcontroller 834 can be, e.g., Part No. CC2541 F128RHAT by Texas Instruments, Inc., of Dallas, Tex., or Part No. nRF51822 by Nordic Semiconductor ASA of Oslo, Norway, or an equivalent part.

In response to a user performing an injection, microcontroller 834 can read medicament information 870 from 1-wire EEPROM 650 of medicament cartridge 600 and transmit medicament information 870 along with injection information, such as, e.g., a determined dosage amount and time and date of the injection, to a recording and/or processing device 872 suitably equipped with, e.g., an antenna 874. Recording and/or processing device 872 can be, e.g., a blood glucose meter. Medicament information 870 can include, e.g., the type of medicament, the expiration date, the batch number, the manufacturer, and/or other appropriate information. In some embodiments, microcontroller 834 can initiate a read of medicament information 870 from 1-wire EEPROM 650 in response to dial 808 returning to its initial (e.g., zero dosage) position, as sensed by position sensor 838, after a user initiates an injection. This prompting of microcontroller 834 to read 1-wire EEPROM 650 can occur, in some embodiments, in a manner similar or identical to that described above in connection with microcontroller 434, Hall effect sensor 438, and rotatable portion 308 of FIG. 4.

FIG. 9 illustrates a method 900 of operating an injection device in accordance with one or more embodiments. At process block 902, method 900 can include receiving an output voltage from a Hall effect sensor. For example, referring to FIG. 4, a user can select a dosage amount by rotating rotatable portion 308 of dial cap 302. Hall effect sensor 438, which rotates with rotatable portion 308 in a magnetic field produced by ring magnet 444, can output a voltage proportional to the angle of rotation of Hall effect sensor 438. In some embodiments, the output voltage of Hall effect sensor 438 can be received by microcontroller 434.

At process block 904, an amount of a dosage based on the received output voltage at process block 902 can be determined. For example, again referring to FIG. 4, a voltage received by microcontroller 434 from Hall effect sensor 438 can be processed by an analog-to-digital converter in microcontroller 434 to convert the received voltage value to a dosage amount. The conversion from a voltage value to a dosage amount can be based on programming code stored in memory 436 and executed in microcontroller 434 that correlates voltage values with dosage amounts.

At process block 906, method 900 can include, in some embodiments, receiving medicament information from a 1-wire EEPROM (electrically erasable programmable read only memory). For example, referring to FIGS. 7 and 8, medicament information stored in 1-wire EEPROM 650 can be received by microcontroller 734 or 834 in response to a user performing an injection with the injection device. The medicament information can include, e.g., the type of medicament, the expiration date of the medicament, the batch number of the medicament, the manufacturer, and/or other information.

At process block 908, method 900 can include transmitting the determined amount of dosage and, in those embodiments where medicament information was received at process block 906, the medicament information. For example, in response to a user performing an injection, microcontroller 834 of FIG. 8 can automatically transmit via, e.g., an internal RF transmitter, injection and medicament information determined by and/or received at microcontroller 834. The injection and medicament information can be transmitted to, in the case of an insulin injection, an appropriately configured blood glucose meter for recording and/or subsequent processing to determine, e.g., a next dosage amount. The transmission can be based on, e.g., ZigBee, BLE (Bluetooth low energy) or ANT wireless technologies. Alternatively, any suitable transmission technology can be used.

The above process blocks of method 900 can be executed or performed in an order or sequence not limited to the order and sequence shown and described. For example, in some embodiments, process block 906 can be performed simultaneously with or before process block 902 and/or 904.

Persons skilled in the art should readily appreciate that the invention described herein is susceptible of broad utility and application. Many embodiments and adaptations of the invention other than those described herein, as well as many variations, modifications, and equivalent arrangements, will be apparent from, or reasonably suggested by, the invention and the foregoing description thereof, without departing from the substance or scope of the invention. For example, although described in connection with insulin pens and the injection of insulin, one or more embodiments of the invention may be used with other types of injection devices and medicaments, such as, e.g., epinephrine. Accordingly, while the invention has been described herein in detail in relation to specific embodiments, it should be understood that this disclosure is only illustrative and presents examples of the invention and is made merely for purposes of providing a full and enabling disclosure of the invention. This disclosure is not intended to limit the invention to the particular apparatus, devices, assemblies, systems or methods disclosed, but, to the contrary, the intention is to cover all modifications, equivalents, and alternatives falling within the scope of the invention.

What is claimed is:

1. A dial cap configured to be attached to a housing of an injection device, the dial cap comprising:
a rotatable portion configured to rotate to select a dosage amount to be delivered by the injection device;
a Hall effect sensor configured to rotate with the rotatable portion and to output a voltage proportional to an angle of rotation of the Hall effect sensor;
a ring magnet encircling the Hall effect sensor; and
a microcontroller configured to:
receive the output voltage of the Hall effect sensor;
determine a dosage amount in response to receiving the output voltage of the Hall effect sensor; and
transmit a determined dosage amount.

2. The dial cap of claim 1 further comprising a printed circuit board attached internally to the rotatable portion and configured to rotate with the rotatable portion, the Hall effect sensor and the microcontroller mounted on the printed circuit board.

3. The dial cap of claim 2 wherein the microcontroller is mounted on a first side of the printed circuit board and the Hall effect sensor is mounted on a second, opposite side of the printed circuit board.

4. The dial cap of claim 1 wherein the microcontroller comprises an analog-to-digital converter operative to receive the output voltage of the Hall effect sensor.

5. An injection device, comprising:
the dial cap of claim 1;
the housing, wherein the housing has a first end and a second end, the dial cap attached to the first end, the housing having therein a plunger and a stored energy source configured to drive the plunger through the second end; and
a cartridge container attached to the second end of the housing, the cartridge container configured to receive a medicament cartridge.

6. The injection device of claim 5 wherein the injection device is an insulin pen configured to deliver insulin to an injection site.

7. The injection device of claim 5, wherein the stored energy source comprises a spring configured to be compressed in response to rotation of the rotatable portion.

8. The injection device of claim 5 further comprising a medicament cartridge received in the cartridge container, the medicament cartridge comprising:
a hollow cylindrical body configured to contain a medicament therein and having an open end;
a piston configured to seal the open end; and
an end cap comprising a 1-wire EEPROM (electrically erasable programmable read only memory) configured to store medicament information therein, the end cap configured to fit over the open end.

9. The injection device of claim 8, wherein:
the end cap further comprises an electrical connection with the 1-wire EEPROM;
the dial cap, the housing, and the cartridge container comprise electrical connections that electrically couples the microcontroller to the 1-wire EEPROM upon receipt of the medicament cartridge in the cartridge container; and
the microcontroller is configured to receive medicament information from the 1-wire EEPROM and transmit the received medicament information.

10. A medicament cartridge configured to be received in a cartridge container of an injection device, the medicament cartridge comprising:
a hollow cylindrical body configured to contain a medicament therein and having an open end;
a piston configured to seal the open end; and
an end cap comprising a 1-wire EEPROM (electrically erasable programmable read only memory) configured to store medicament information therein, the end cap configured to fit over the open end.

11. The medicament cartridge of claim 10 wherein the 1-wire EEPROM has stored therein medicament information including at least one of a type of medicament, an expiration date, and a batch number.

12. The medicament cartridge of claim 10 wherein the hollow cylindrical body contains insulin therein.

13. An injection device, comprising:
a dial cap comprising a microcontroller and configured to rotate to select a dosage amount to be delivered by the injection device;
a housing having a first end and a second end, the dial cap attached to the first end, the housing having therein a plunger and a stored energy source configured to drive the plunger through the second end;
a cartridge container attached to the second end of the housing, the cartridge container configured to receive a medicament cartridge; and
the medicament cartridge of claim 10 received in the cartridge container.

14. The injection device of claim 13 wherein:
the end cap of the medicament cartridge further comprises an electrical contact in communication with the 1-wire EEPROM; and
the dial cap, the housing, and the cartridge container comprise an electrical connection that electrically couples the microcontroller to the electrical contact of the end cap.

15. The injection device of claim 13 wherein the dial cap further comprises a Hall effect sensor configured to rotate and output a voltage proportional to an angel of rotation.

16. A method of operating an injection device, the method comprising:
receiving an output voltage from a Hall effect sensor located in the injection device;
determining an amount of a dosage based on the received output voltage;
receiving medicament information from a 1-wire EEPROM (electrically erasable programmable read only memory) located in a medicament cartridge of the injection device; and
transmitting the determined amount of the dosage and the medicament information to a device external to the injection device.

17. The method of claim 16 further comprising electrically coupling the 1-wire EEPROM to a microcontroller of the injection device.

18. The method of claim 16 further comprising:
providing a dial cap comprising:
a rotatable portion configured to rotate to select a dosage amount to be delivered by the injection device;
the Hall effect sensor configured to rotate with the rotatable portion;
a ring magnet encircling the Hall effect sensor; and
a microcontroller configured to:
receive the output voltage of the Hall effect sensor;
determine the amount of the dosage in response to receiving the output voltage of the Hall effect sensor; and
transmit the determined amount of the dosage.

19. The method of claim 16 further comprising providing insulin in the medicament cartridge.

20. The method of claim 16 further comprising:
providing the medicament cartridge comprising:
a hollow cylindrical body configured to contain a medicament therein and having an open end;
a piston configured to seal the open end; and
an end cap comprising the 1-wire EEPROM, the end cap configured to fit over the open end.
